(19) United States
(12) Patent Application Publication
(10) Pub. No.: US 2008/0274057 A1
(43) Pub. Date: Nov. 6, 2008

(54) Staudinger Reaction in Imaging and Therapy and Kits for Use in Imaging and Therapy

(75) Inventors: Marc Stefan Robillard, Eindhoven (NL); Holger Gruell, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/089,083

(22) PCT Filed: Oct. 2, 2006
(86) PCT No.: PCT/IB2006/053584

§ 371 Date: Apr. 3, 2008

(30) Foreign Application Priority Data
Oct. 4, 2005 (EP) 05109198.1

(51) Int. Cl. A61K 49/00; A61K 39/395

(52) U.S. Cl. 424/9.1; 424/130.1

(57) ABSTRACT

The Staudinger reaction can be used for activation of prodrugs or pro-imaging probes. The invention relates to a method of preparing and activating prodrugs or pro-imaging probes by using the Staudinger reaction and to kits for medical imaging and/or therapy comprising at least one prodrug and/or pro-imaging probe comprising at least one azide and/or phosphine group.

### FIELD OF THE INVENTION

The present invention relates to novel methods, kits and compounds, for use in medical imaging and therapy. The present invention also relates to novel compounds and kits for pre-localized imaging and/or therapy and to methods of production and use thereof.

### BACKGROUND TO THE INVENTION

In the medical area the use of inactive compounds such as prodrugs and inactivated imaging probes which are activated in a specific site in the human or animal body is well known. Also targeted delivery of actives such as prodrugs and imaging agents has been studied extensively. Much effort has been devoted to drug delivery systems that effect drug release selectivity at a target site and/or at a desired moment in time. One way is to selectively activate a (systemic) prodrug specifically by local and specific enzymatic activity.

However, in many cases a target site of interest lacks a suitable overexpressed enzyme. A promising solution that has been suggested in the art is to transport an enzyme to target tissue via a technique called antibody-directed enzyme prodrug therapy (ADEPT). In this approach an enzyme is targeted to a tumor site by conjugation to an antibody that binds a tumor-associated antigen. After systemic administration of the conjugate, its localization at the target and clearance of unbound conjugate, a designed prodrug is administered systemically and locally activated. Similar techniques are called polymer-directed enzyme prodrug therapy (PDEPT), macromolecular-directed enzyme prodrug therapy (MDEPT), virus-directed enzyme prodrug therapy (VDEPT) and gene-directed enzyme prodrug therapy (GDEPT). In all cases an exogenous enzyme is delivered to or expressed in the target tissue, in order to locally activate a (systemic or targeted) prodrug.

There are various drawbacks to the known techniques of enzymatically activated prodrugs. In the method where an enzyme is delivered to the target site this requires the catalysis of a reaction that must not be accomplished by an endogenous enzyme. Enzymes of non-mammalian origin that meet these needs are likely to be highly immunogenic, a fact that makes repeated administration impossible. In order to suppress antibody responses against the enzyme-antibody conjugate, the immunosuppressive agent cyclosporin has been used. However, use of immunosuppression in cancer is not desirable and offers only limited mitigation of the immune response.

Furthermore, enzymes may not be able to reach the intracellular space in the case their action is desired to take place there. In poorly vascularized and/or dense tumors, delivery of large enzyme-antibody conjugates is restricted and not all cells are reached. Furthermore, these large conjugates have slow kinetics/targeting speeds, and poor clearance from non-target tissues.

Similar problems may exist in the field of molecular imaging with selectively activeatable probes. The lack of a suitable over-expressed enzyme invites the use of targeted exogenous enzymes for the local activation of pro-imaging probes. However, the extension of the ADEPT concept to molecular imaging would be hampered by the same needs and drawbacks discussed above.

It is an object of the invention to overcome one or more of the above-outlined drawbacks.

### SUMMARY OF THE INVENTION

The present invention provides prodrugs and pro-imaging probes, kits of these, methods of producing and activating such probes and prodrugs, and methods of applying probes and prodrugs in the context of medical imaging and therapy.

In its broadest aspect, the present invention relates to two components that interact with each other to trigger the release or activation of a drug and/or imaging agent. These components are of use in medical imaging and therapy, more particularly in targeted imaging and therapy and in methods where a pre-localized prodrug/pro-imaging probe or activator is used.

According to the invention the trigger is obtained by the Staudinger reaction, and each of the components of the invention comprise a reaction partner for the Staudinger reaction, i.e. a phosphine or an azide group, respectively.

In a first aspect the invention relates to a method for preparing and activating a prodrug or a pro-imaging probe, the method comprising the steps of:

a) functionalising a drug (x) or imaging probe (y) with at least one azide and/or phosphine group to create a prodrug or pro-imaging probe;
b) reacting the prodrug or pro-imaging probe by a Staudinger reaction with a composition (z) comprising at least one azide and/or phosphine group as a reaction partner in the Staudinger reaction,
thereby activating the drug or imaging probe.

In a further aspect the invention relates to a kit suitable for use in medical imaging or therapeutics.

In another aspect the invention relates to use of a prodrug or pro-imaging probe comprising an azide and/or a phosphine group, said phosphine and/or said azide groups being suitable reaction partners for the Staudinger reaction, as a tool in targeted medical imaging or in the manufacture of a tool for medical imaging.

Furthermore the invention relates to a prodrug or pro-imaging probe according to the invention for use in the preparation of a medicament.

### DETAILED DESCRIPTION OF THE INVENTION

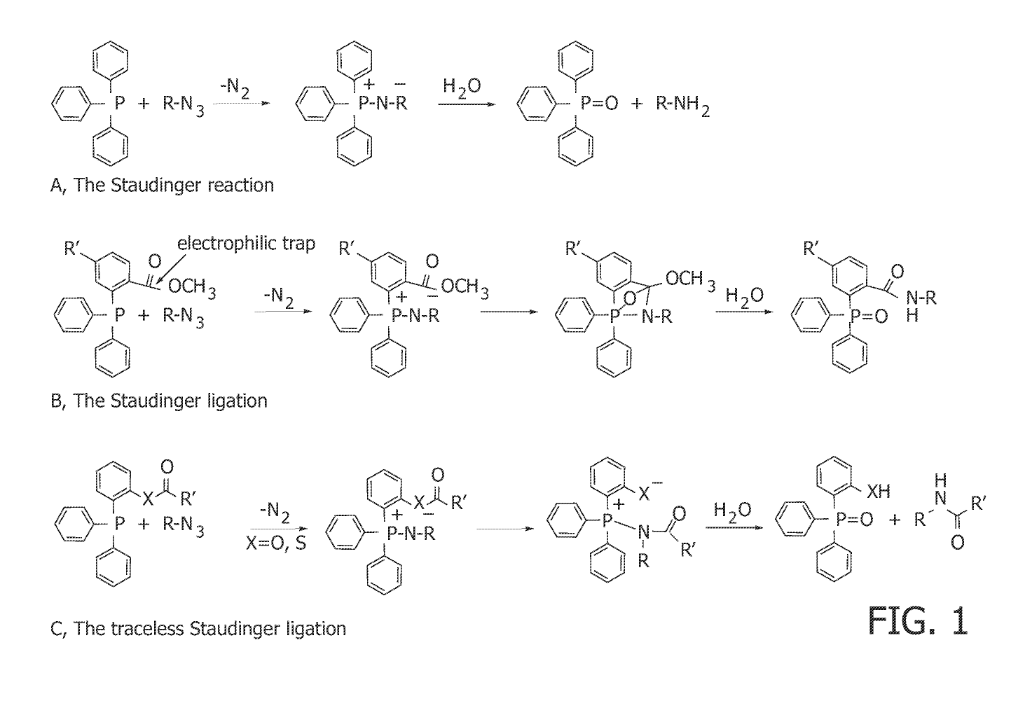

FIG. 1 shows the Staudinger reaction (A) and the Staudinger ligation in two embodiments (B, C).

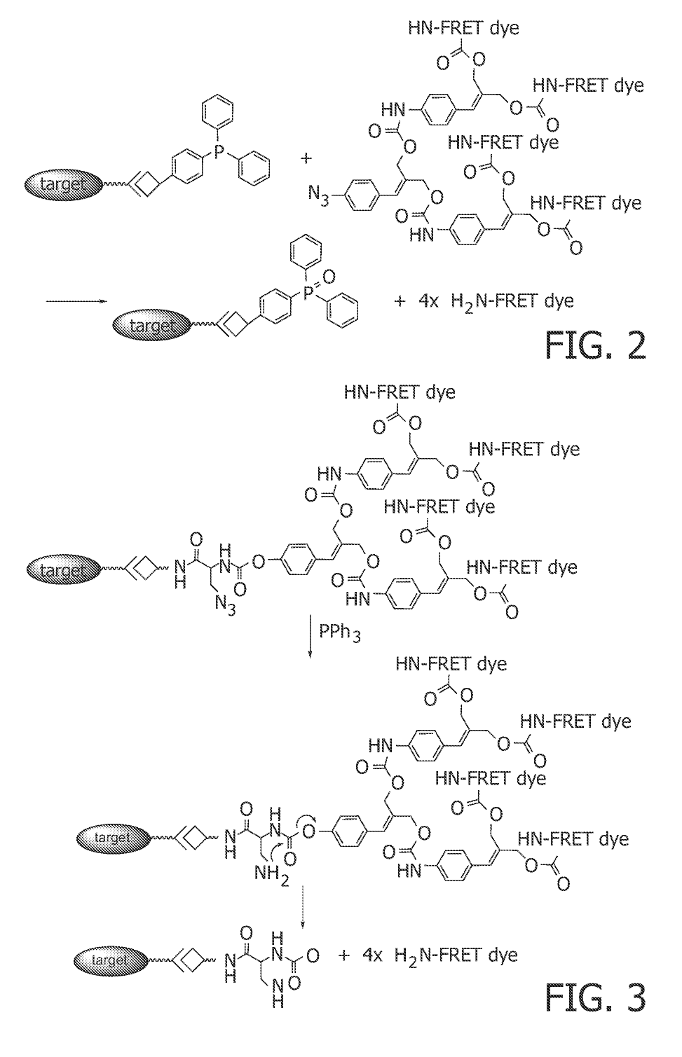

FIG. 2 illustrates example 1 wherein triphenylphosphine conjugates are targeted to a disease site and react with azido-trigger functionalized cascade release dendrimers containing multiple FRET dyes, thereby activating and releasing the dye.

FIG. 3 illustrates example 2 wherein azido-trigger functionalised cascade release dendrimers containing multiple FRET dyes are targeted to a disease site. Subsequent administration of triphenylphosphine leads to activation of the dye.

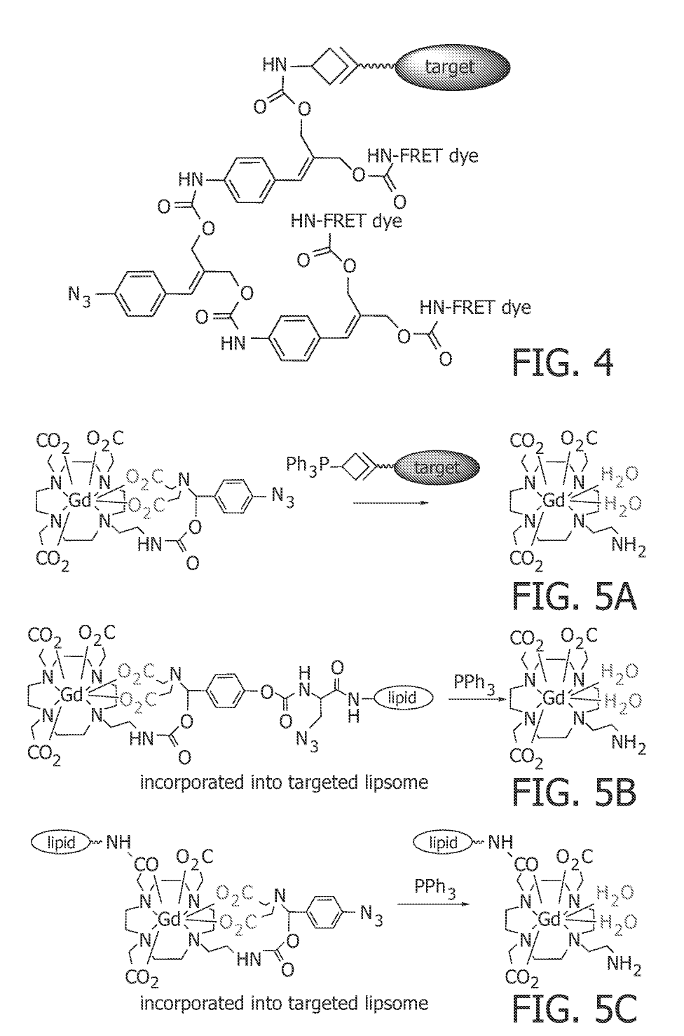

FIG. 4 illustrates example 3 wherein as an alternative to example 2, the targeting moiety is conjugated to the dendrimer via one of its tail ends instead of one FRET dye.

FIG. 5 illustrates example 4 wherein in (A) an MRI-inactive Gd chelate is functionalised with an azide, and reacts with prelocalized triphenylphosphine in a Staudinger reaction to release active Gd chelate. In (B) a targeted and prelocalized liposome with appended MRI-inactive Gd chelate is activated after reaction of the azide group on the chelate with systemic triphenylphosphine in a Staudinger reaction. In (C) the lipid tail is attached to a position that allows activation but not release of the MRI probe after the Staudinger reaction.

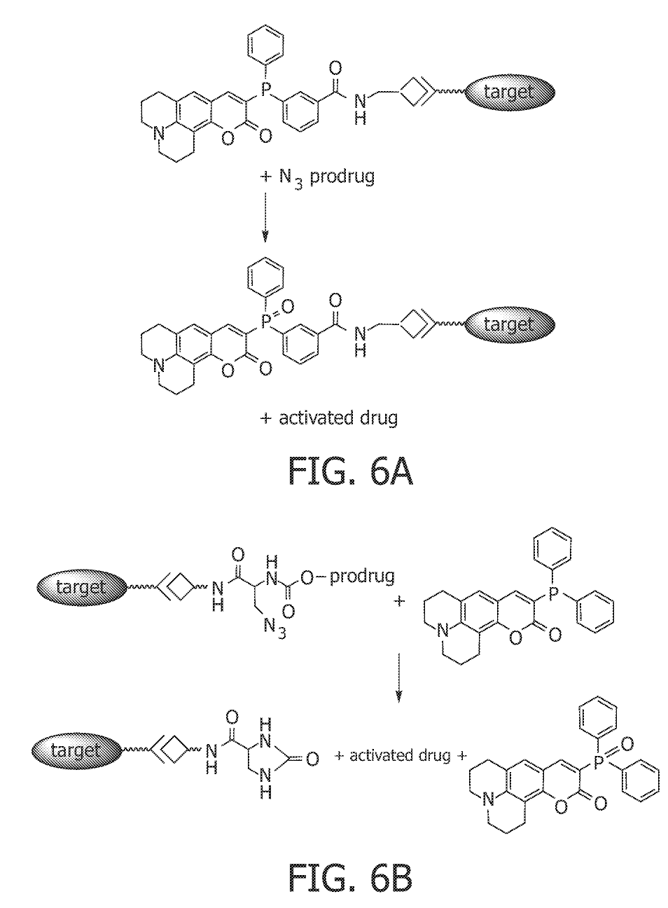

FIG. 6 illustrates example 7, wherein example A uses a prelocalized profluorescent triphenylphosphine dye that is activated by the Staudinger reaction with an azide prodrug, leading to active drug molecules and activation of the fluorescent probe. In example B, azide prodrug is targeted to the disease site and activated by the profluorescent triphenylphosphine dye, simultaneously effecting activation of the dye.

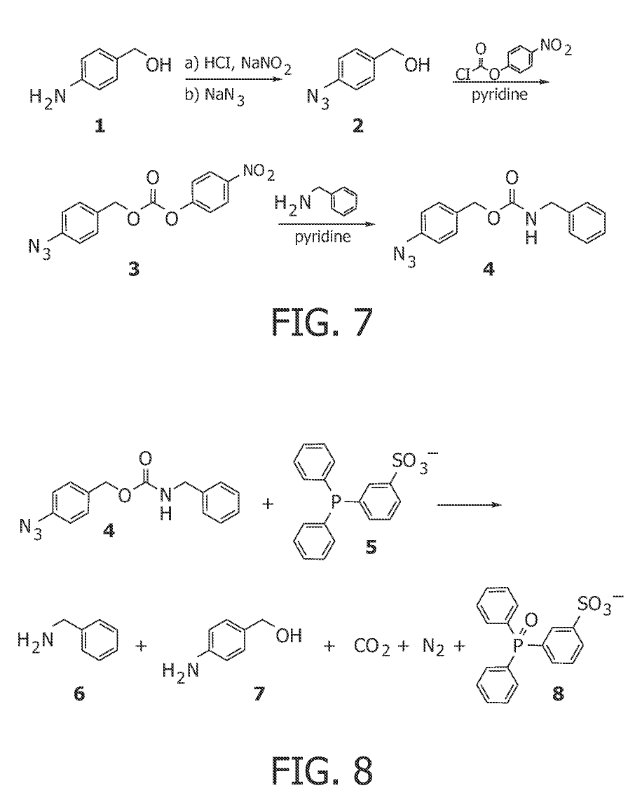

FIG. 7 illustrates example 8, wherein the model azido-prodrug 4-azidobenzyl N-benzyl carbamate (4) is synthesized in 3 steps.

FIG. 8 illustrates example 9, wherein the model azido-prodrug 4-azidobenzyl N-benzyl carbamate (4) is activated by 3-(diphenylphosphino)benzenesulfonate.

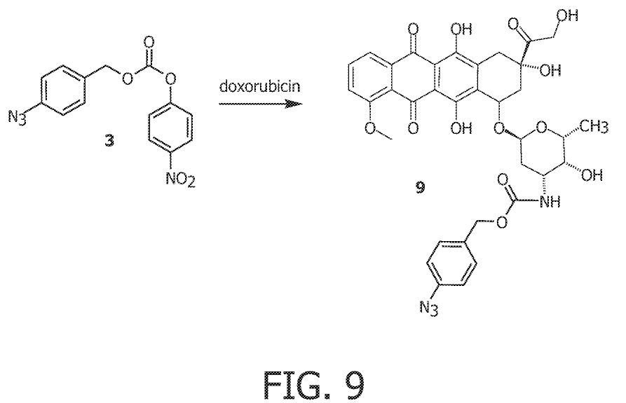

FIG. 9 illustrates the Synthesis of doxorubicin prodrug as described in example 10.

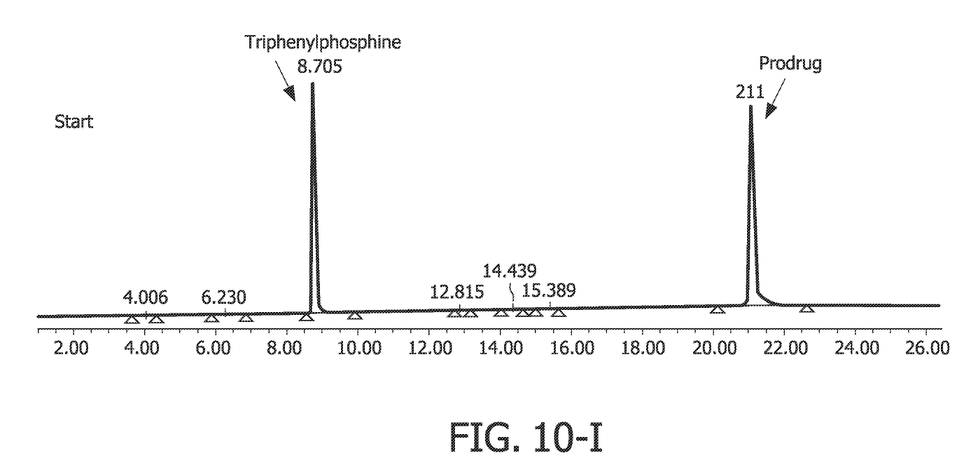

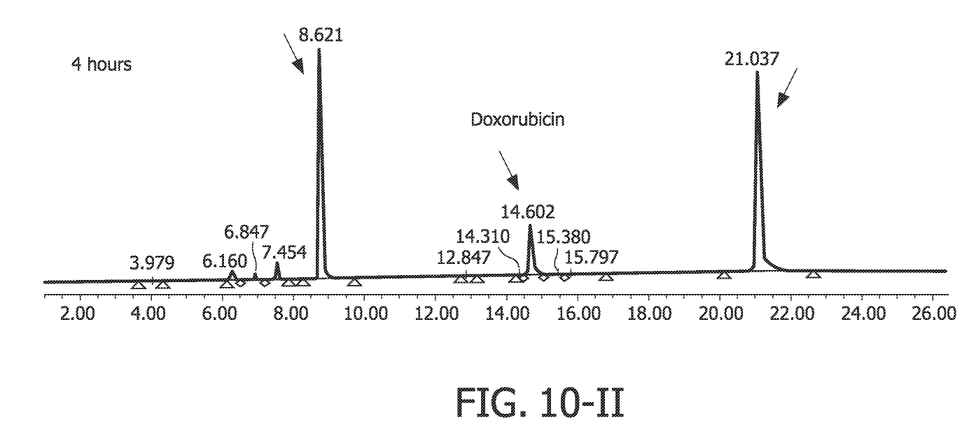

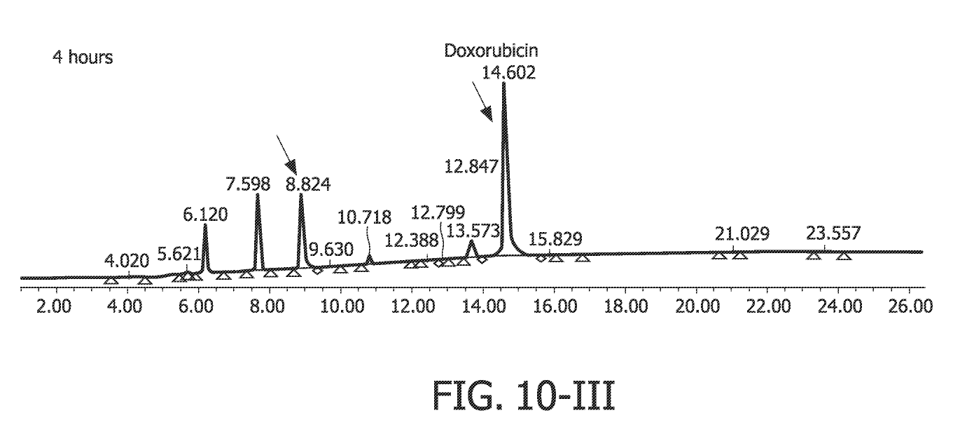

FIGS. 10-12 illustrate the activation of the doxorubicin prodrug (9) followed with HPLC and LC-MS, described in more detail in example 11.

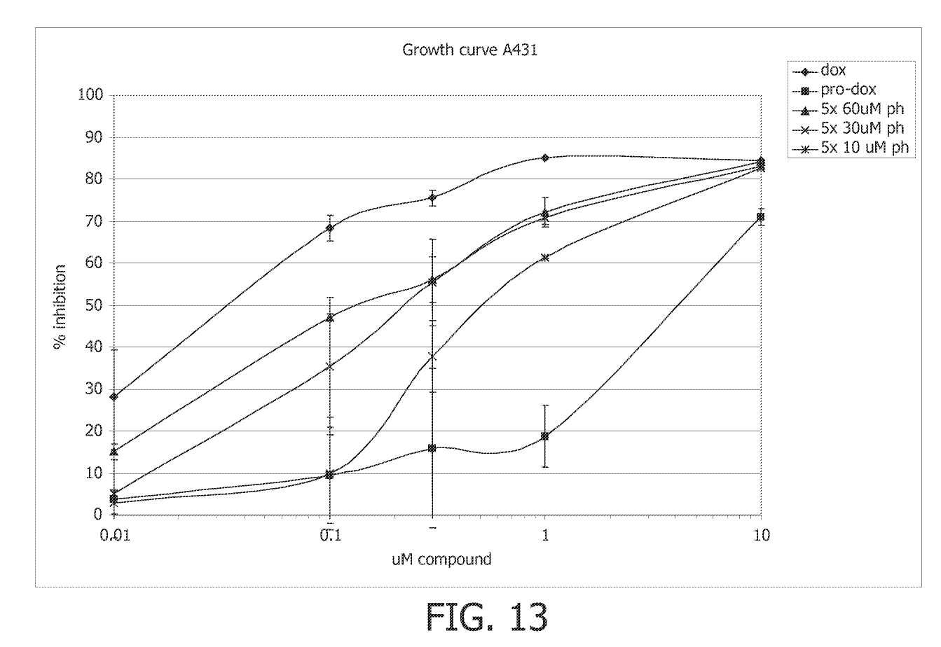

FIGS. 13-15 illustrate the cell proliferation assay using A431 cells with prodrug 9 (pro-dox) in situ activated by triphenylphosphine as described in example 12.

### DETAILED DESCRIPTION OF THE INVENTION

In the current invention, use is made of the Staudinger reaction (Gololobov et al, Tetrahedron 1981, vol 37, pages 437-472).

To avoid any possible confusion, in the context of the invention the Staudinger reaction is not the same as the Staudinger ligation. The Staudinger reaction occurs between a phosphine and an azide to produce an aza-ylide. In the presence of water, this intermediate hydrolyses spontaneously to yield a primary amine and the corresponding phosphine oxide. In the Staudinger ligation the hydrolysis of the aza-ylide is prevented by introduction of an electrophylic trap on the phosphine, leading to the generation of a stable, covalent bond between the two reaction partners. Contrary to this ligation, the current invention relates to the Staudinger reaction wherein the hydrolysis leads to the formation of an active drug or active imaging probe. The Staudinger ligation is for example described in Lemieux et al, J. Am. Chem. Soc. 2003, 125, 4708-4709 and in Prescher et al, Nature vol 430, 19 Aug. 2004, 783-877. The differences between the Staudinger ligation and the Staudinger reaction are illustrated in FIG. 1. Generally in the context of the invention, prior to the Staudinger reaction taking place the drug or probe is in the form of a (partly) inactivated prodrug or (partly) inactivated pro-imaging probe. The (partial) inactivation of a drug or imaging probe is also referred to as “masking” of the drug or probe.

Embodiments of the present invention provide a chemical reaction wherein the two participating functional groups are much smaller than an enzyme. With the methods of the present invention, two participating functional groups, e.g. azide and phosphine, are used which equal the tremendous selectivity of non-covalent recognition events that occur in many biological processes, such as enzyme-substrate interaction. In accordance with an aspect of the present invention two participating functional groups are selected that have a finely tuned reactivity so that interference with coexisting functionality is avoided. In accordance with a further aspect of the present invention reactive partners are selected which are abiotic and recognize only each other while ignoring their cellular/physiological surroundings, i.e. they are bio-orthogonal. The demands on selectivity imposed by a biological environment preclude the use of most other conventional reactions.

The kits and method of the invention are very suitable for use in targeted delivery of drugs and/or imaging probes.

Moreover, the present invention is particularly suitable for use in multimodal imaging, optionally using different imaging agents to visualize the same target.

A “primary target” as used in the present invention relates to a target to be detected by imaging or a target for therapy. For example, a primary target can be any molecule, which is present in an organism, tissue or cell. Targets for imaging include cell surface targets, e.g. receptors, glycoproteins; structural proteins, e.g. amyloid plaques; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof. Examples of primary targets include compounds such as proteins of which the presence or expression level is correlated with a certain tissue or cell type or of which the expression level is up regulated or down-regulated in a certain disorder. According to a particular embodiment of the present invention, the primary target is a protein such as a receptor. Alternatively, the primary target may be a metabolic pathway, which is up regulated during a disease, e.g. infection or cancer, such as DNA synthesis, protein synthesis, membrane synthesis and saccharide uptake. In diseased tissues, above-mentioned markers can differ from healthy tissue and offer unique possibilities for early detection, specific diagnosis and therapy especially targeted therapy.

A “targeting probe” as used herein refers to a probe, which binds to the primary target. The targeting probe comprises a “primary targeting moiety” and a “Staudinger reaction partner”.

A “Staudinger reaction partner” is a moiety which is selected from the group comprising azide and phosphine which may react with another Staudinger reaction partner in the Staudinger reaction. In specific embodiments, the Staudinger reaction partner will be one or more azide groups. However, in other particular embodiments, applications are envisaged wherein the Staudinger reaction partner will be one or more phosphine groups.

A “primary targeting moiety” as used in the present invention relates to the part of the targeting probe or prodrug or pro-imaging probe, which binds to a primary target. Particular examples of primary targeting moieties are peptides or proteins, which bind to a receptor. Other examples of primary targeting moieties are antibodies or fragments thereof, which react with a cellular compound. Antibodies can be raised to non-proteinaceous compounds as well as to proteins or peptides. Other primary targeting moieties can be made up of aptamers, oligopeptides, oligonucleotides, oligosacharides, as well as peptoids and organic drug compounds. A primary targeting moiety preferably binds with high specificity, with a high affinity and the bond with the primary target is preferably stable within the body.

“Building blocks” are defined as molecules that are involved in pathways in a cell such as metabolic pathways. Building blocks may form part of molecules that are present in the cell such as sugars, DNA, RNA, peptides, proteins. Metabolic tracers and precursors are also referred to as building blocks. Examples of building blocks are glucose, nucleo bases, amino acids, fatty acids, acetate and choline.

Nucleo bases are the parts of RNA and DNA that are involved in pairing up. A nucleobase covalently bound to the 1′ carbon of a ribose or deoxyribose is called a nucleoside, and a nucleoside with one or more phosphate groups attached at the 5′ carbon is called a nucleotide. Examples of nucelobases are thymine, uracil, guanine, cytosine.

The “imaging probe” comprises a detectable label, such as for instance a contrast providing unit.

The term “pro-imaging probe” relates to a composition which comprises a detectable label that is suitable for use in imaging and which is functionalised with an azide and/or a phosphine group. This functionalisation may lead to partial or whole inactivation of the label.

An “activator” refers to a compound (z) that reacts with that part of the pro-imaging probe or prodrug that comprises a reaction partner for the Staudinger reaction. In a particular embodiment the activator will comprise one or more phosphine groups.

A “detectable label” as used herein relates to the part of the imaging probe that allows detection of the probe, e.g. when present in a cell, tissue or organism. One type of detectable label envisaged within the context of the present invention is a contrast providing agent. Different types of detectable labels are envisaged within the context of the present invention and are described herein.

A “therapeutic probe” as used herein refers to a probe comprising a pharmaceutically active compound, such as but not limited to a therapeutic compound. Examples of pharmaceutically active compounds are provided herein. A therapeutic probe can optionally also comprise a detectable label.

The term “prodrug” relates to a composition which comprises a therapeutic or pharmacologically active moiety that is functionalised with an azide and/or a phosphine group. This functionalisation may lead to partial or whole inactivation of the drug.

The term “isolated” as used herein refers to a compound being present outside the body or outside a cell or fraction of cell, e.g. cell lysate. With respect to particular features attributed to an isolated probe or combined probe, e.g. a primary targeting probe, imaging probe or a therapeutic probe or a combination thereof, in the context of the present invention, this refers to a probe as present outside the human or animal body, tissue or cell. It does not refer to conjugates which are formed within a body, tissue or cell after the consecutive addition of the constituent components of said conjugate to said body tissue or cell.

A particular aspect of the present invention relates to localized imaging or therapy methods. This aspect requires the separate use of two components of the present invention and relates to the separation in time of the administration to the patient of the component which comprises the targeting moiety or ensures the targeting by being a substrate of a particular reaction, and the component which ensures the image or therapeutic effect. The time in between administration of the two components can vary but generally ranges from about 10 minutes to several hours or even days.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

It is furthermore to be noticed that the term “comprising”, used in the description and in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

The invention in a first aspect relates to a method for preparing and activating a prodrug or pro-imaging probe.

In step (a) of the method according to the invention a drug (x) or imaging probe (y) is functionalised with at least one azide and/or phosphine group to create a prodrug or pro-imaging probe.

In general the prodrug or pro-imaging probe, comprises at least one azide and/or phosphine group but they may optionally comprise at least 2 or more, at least 3 or more, at least 4 or more, or at least 5 or more of these substituents. Preferably the amount of azide and/or phosphine substituents is from 1 to 50, more preferred from 1 to 30, even more preferred from 2 to 10 per prodrug or pro-imaging probe molecule.

According to one embodiment, an amine group of an active drug or imaging probe is converted to an azide. This modification may lead to (partial) inactivation of the drug or imaging probe functionality.

According to another embodiment, e.g. where the drug or imaging probe does not contain a suitable amine group that can be converted to an azide, these molecules are modified with an azide funtionalised trigger-linker system [Papot et al., Curr. Med. Chem.-Anti-cancer agents 2002, 2, 155-185]. Methods to introduce an azide group are for example disclosed in WO-A-03/003806.

Also drugs and probes that contain an hydroxyl moiety may be modified using a linker system to accommodate an azide-trigger.

The azide or phosphine group may be a partner in the Staudinger reaction with its Staudinger counterpart. Hence in a second step (b) the prodrug or pro-imaging probe is activated by a Staudinger reaction with a composition (z) comprising at least one azide and/or phosphine group as a reaction partner in the Staudinger reaction, thereby activating the drug or imaging probe.

For the embodiment where a pro-drug or pro-imaging probe comprises at least one azide and at least one phosphine group, it will be appreciated that the positioning or masking of these respective reactive groups is preferably such that they do not prematurely react intra- or intermolecularly.

In one embodiment, where the prodrug or pro-imaging probe comprises an aromatic azide moiety, a Staudinger reaction with a triphenylphosphine affords the aniline derivative that initiates an eliminating cascade that releases a drug or imaging probe, thereby activating the drug or imaging probe.

In a preferred embodiment, the prodrug or pro-imaging probe is essentially inactivated due to the modification in step (a). Preferably the subsequent Staudinger reaction of step (b) leads to activation of the drug or imaging probe. Therefore the composition (z) is in this specification also referred to as activator.

In the context of the invention, the activation in step (b) is to be interpreted broadly by a skilled person. Even a slight improvement in activity is considered activation in this context. In this context the term “activation” not only covers embodiments wherein activity is increased in a certain amount but also mere release of a drug or imaging probe which may have been active before the release. A suitable synonym to activation in this context is “unmasking”. In an example of unmasking the activation converts a previously non-toxic composition into a (locally) toxic composition. It will therefore be appreciated that the label or drug that is functionalised with a moiety that is a partner in the Staudinger reaction, is also referred to as an activatable label or drug, although the label/drug may be in a detectable, respectively active state before the activation.

Optionally, the above-described activation through use of the Staudinger reaction is combined with cascade-release dendrimers, allowing the release and activation of multiple drugs or probes.

Without wishing to be bound by any theory it is believed that the Staudinger reaction between an azide and phosphine of which at least one is located on a pro-drug or pro-imaging probe, triggers the release of an active drug or detectable label. This triggering may be tuned such that the release occurs at a desired moment in time and/or at a desired spot.

The composition (z) that reacts with the azide and/or phosphine functionality on the prodrug or pro-imaging probe, may be any suitable compound with an azide or phosphine group that may take part in the Staudinger reaction, leading to activation of drug and/or imaging probe. In a preferred embodiment composition (z) is a targeting probe comprising a primary targeting moiety and a Staudinger reaction partner.

Composition (z) preferably comprises at least one azide and/or phosphine group but may optionally comprise at least 2 or more, at least 3 or more, at least 4 or more, or at least 5 or more of these substituents. Preferably the amount of azide and/or phosphine substituents is from 1 to 50, more preferred from 1 to 30, even more preferred from 2 to 10 per composition (z) molecule.

It is highly preferred that the composition (z) comprises at least 2 or more azide and/or phosphine groups.

The properties of the phosphine moiety, preferably triphenylphosphine moiety, that is linked to prodrug (x), pro-imaging probe (y) or composition (z) may be tuned in a manner well known in the art. For example solubility and distribution or reactivity may be influenced by introduction of appropriate functional groups (e.g. SO₃) on the aromatic rings of a triphenylphosphine moiety.

According to one embodiment of the present invention, the phosphine is represented by P(R1, R2, R3) wherein each of R1, R2 and R3 are linked to P. In a preferred embodiment, R1, R2 and R3 are aryl groups, including substituted aryl groups or cycloalkyl groups, e.g. cyclohexyl groups.

R1, R2 and R3 may be the same or different.

Optionally R1, R2 and/or R3 is used as a linker to a drug or imaging probe, or composition (z), or primary targeting moiety to form a prodrug or pro-imaging probe or composition (z) according to the invention.

In a further preferred embodiment, use is made of a combination of a prodrug and a (pro)-imaging probe. In this embodiment, the administration and presence/activation of a drug may be monitored via the imaging probe.

Hence optionally the prodrug is linked to an imaging probe comprising a label and at least one of the prodrug and the imaging probe unit comprises a phosphine and/or an azide group.

In an alternative embodiment, the prodrug comprises an azide and/or phosphine group while the imaging probe comprises the other partner of the Staudinger reaction, i.e. at least an azide and/or a phosphine group. In this embodiment, composition (z) is optional.

In this embodiment, the invention relates to a method for preparing and activating a prodrug and/or a pro-imaging probe comprising the steps of:

a) functionalising a drug (x) with at least one azide and/or phosphine group to create a prodrug;
b) functionalising an imaging probe (y) with at least one azide and/or phosphine group which is a partner in the Staudinger reaction for the azide and/or phosphine group of the prodrug of step (a) to create a pro-imaging probe;
c) reacting the prodrug and pro-imaging probe by a Staudinger reaction, thereby activating the drug and/or imaging probe.

In a further aspect the invention relates to a kit for medical imaging or therapeutics with an imaging probe or a drug, comprising:

at least one prodrug (x) and/or pro-imaging probe (y) comprising at least one azide and/or phosphine group;

a composition (z) comprising at least one azide and/or phosphine group capable of reacting with the prodrug or pro-imaging probe in a Staudinger reaction to form the drug or imaging probe.

The above-described preferred embodiments for the prodrug, the pro-imaging probe, the composition (z) and their application likewise apply to the components of this kit.

Preferably the kit comprises at least one prodrug.

In an even more preferred embodiment, the kit comprises a prodrug and a pro-imaging probe. This enables monitoring of drug release/activation and accumulation of drug and therewith provides useful information on drug distribution and activity.

Preferably the prodrug and the pro-imaging probe comprise a phosphine group. In this embodiment the composition (z) comprises an azide group which may react with the phosphine groups in a Staudinger reaction.

Alternatively, the prodrug and the pro-imaging probe each comprise different reactive group selected from phosphine and azide. Thus according to this embodiment, the prodrug and the pro-imaging probe are partners in the Staudinger reaction. In this embodiment, the kit preferably further comprises instructions about sequential administration of the prodrug and the pro-imaging probe. In this embodiment, the presence of composition (z) is optional.

Therefore in another aspect the invention relates to a kit for targeted medical imaging and/or therapeutics comprising:

at least one prodrug (x) comprising at least one azide and/or phosphine group;

at least one pro-imaging probe (y) comprising at least one azide and/or phosphine group capable of reacting with the prodrug in a Staudinger reaction to form a drug and an imaging probe.

The azide- or phosphine-comprising targeting, imaging and therapeutic probes of the present invention are biocompatible and can be administered in an identical or similar way as conventional molecules which are currently used in medical imaging or therapy. In addition, the detectable labels of the imaging probe are known to the skilled person and require conventional methodology and apparatus.

To enable targeting of the drug, imaging probe or composition (z), the prodrug (x), pro-imaging probe (y) or composition (z) preferably comprise a targeting moiety, which is referred to as a “primary targeting moiety”. This moiety suitably binds to a primary target which is the target to be either treated by therapy or to be detected by imaging. Optionally at least 2 of x, y, z or each of x, y, z comprise a targeting moiety.

According to one embodiment, the invention is used for targeted imaging or targeted therapy. According to this embodiment, imaging of specific primary target is achieved by specific binding of a primary targeting moiety and detection of this binding using detectable labels which are activated by the Staudinger reaction.

According to the present invention, the primary target can be selected from any suitable targets within the human or animal body or on a pathogen or parasite, e.g. a group comprising cells such as cell membranes and cell walls, receptors such as cell membrane receptors, intracellular structures such as Golgi bodies or mitochondria, enzymes, receptors, DNA, RNA, viruses or viral particles, antibodies, proteins, carbohydrates, monosacharides, polysaccharides, cytokines, hormones, steroids, somatostatin receptor, monoamine oxidase, muscarinic receptors, myocardial sympatic nerve system, leukotriene receptors, e.g. on leukocytes, urokinase plasminogen activator receptor (uPAR), folate receptor, apoptosis marker, (anti-)angiogenesis marker, gastrin receptor, dopaminergic system, serotonergic system, GABAergic system, adrenergic system, cholinergic system, opoid receptors, GPIIb/IIIa receptor and other thrombus related receptors, fibrin, calcitonin receptor, tuftsin receptor, integrin receptor, VEGF/EGF receptors, matrix metalloproteinase (MMP), P/E/L-selectin receptor, LDL receptor, P-glycoprotein, neurotensin receptors, neuropeptide receptors, substance P receptors, NK receptor, CCK receptors, sigma receptors, interleukin receptors, herpes simplex virus tyrosine kinase, human tyrosine kinase.

In order to allow specific targeting of the above-listed primary targets, the targeting moiety can comprise compounds including but not limited to antibodies, antibody fragments, e.g. Fab2, Fab, scFV, VHH, proteins, peptides, e.g. octreotide and derivatives, VIP, MSH, LHRH, chemotactic peptides, bombesin, elastin, peptide mimetics, carbohydrates, monosacharides, polysaccharides, viruses, drugs, polymers, chemotherapeutic agents, receptor agonists and antagonists, cytokines, hormones, steroids. Examples of organic compounds envisaged within the context of the present invention are, or are derived from, estrogens, e.g. estradiol, androgens, progestins, corticosteroids, methotrexate, folic acid, and cholesterol. In a preferred embodiment, the primary targeting moiety is an antibody.

According to a particular embodiment of the present invention, the primary target is a receptor and suitable primary targeting moieties include but are not limited to, the ligand of such a receptor or a part thereof which still binds to the receptor, e.g. a receptor binding peptide in the case of receptor binding protein ligands.

Other examples of primary targeting moieties of protein nature include interferons, e.g. alpha, beta, and gamma interferon, interleukins, and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin.

Alternative examples of primary targeting moieties include DNA, RNA, PNA and LNA which are e.g. complementary to the primary target.

According to a particular embodiment of the invention, small lipophilic primary targeting moieties are used which can bind to an intracellular primary target.

According to a further particular embodiment of the invention, the primary target and primary targeting moiety are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting primary targets with tissue-, cell- or disease-specific expression. For example, membrane folic acid receptors mediate intracellular accumulation of folate and its analogs, such as methotrexate. Expression is limited in normal tissues, but receptors are overexpressed in various tumor cell types.

According to one embodiment, the primary targeting moiety and the imaging probe and/or the therapeutic probe or composition (z) can be multimeric compounds, comprising a plurality of primary targeting moieties and/or Staudinger reaction partners and/or drugs/imaging probes, preferably a plurality of primary targeting moieties. These multimeric compounds can be polymers, dendrimers, liposomes, polymer particles, or other polymeric constructs.

In a further aspect the invention relates to the use of a prodrug or pro-imaging probe comprising an azide and/or a phosphine group, said phosphine and/or said azide groups being suitable reaction partners for the Staudinger reaction, as a tool in medical imaging, preferably as a tool in targeted medical imaging.

The invention further relates to the use of a pro-imaging probe comprising an azide and/or a phosphine group, said phosphine and/or said azide groups being suitable reaction partners for the Staudinger reaction, in the manufacture of a tool for medical imaging.

The invention further relates to the use of a prodrug comprising an azide and/or phosphine group and a detectable label, said phosphine and/or azide groups being suitable reaction partners for the Staudinger reaction, in the manufacture of a tool for medical imaging.

According to a particular embodiment of the present invention, the compounds and methods of the present invention are used for imaging, especially medical imaging. Use is made of an imaging probe comprising one or more detectable labels. Particular examples of detectable labels of the imaging probe are contrast agents used in traditional imaging systems such as MRI-imageable agents, spin labels, optical labels, ultrasound-responsive agents, X-ray-responsive agents, radionuclides, (bio)luminescent and FRET-type dyes. Exemplary detectable labels envisaged within the context of the present invention include, and are not necessarily limited to, fluorescent molecules, e.g. autofluorescent molecules, molecules that fluoresce upon contact with a reagent, etc., radioactive labels, imaging agents for MRI comprising paramagnetic metal, imaging reagents, e.g., those described in U.S. Pat. Nos. 4,741,900 and 5,326,856) and the like.

The MRI-imageable agent can be a paramagnetic ion or a superparamagnetic particle. The paramagnetic ion can be an element selected from the group consisting of Gd, Fe, Mn, Cr, Co, Ni, Cu, Pr, Nd, Yb, Tb, Dy, Ho, Er, Sm, Eu, Ti, Pa, La, Sc, V, Mo, Ru, Ce, Dy, Tl. A particular embodiment of the present invention relates to the use of “smart” or “responsive” MRI contrast agents, as described more in detail hereafter.

The ultrasound responsive agent can comprise a microbubble, the shell of which comprise a phospholipid, and/or (biodegradable) polymer, and/or human serum albumin. The microbubble can be filled with fluorinated gasses or liquids.

The X-ray-responsive agents include but are not limited to Iodine, Barium, Barium sulfate, Gastrografin or can comprise a vesicle, liposome or polymer capsule filled with Iodine compounds and/or barium sulfate.

Moreover, detectable labels envisaged within the context of the present invention also include peptides or polypeptides that can be detected by antibody binding, e.g., by binding of a detectable labeled antibody or by detection of bound antibody through a sandwich-type assay.

In one embodiment the detectable labels are small size organic PET and SPECT labels, such as ¹⁸F, ¹¹C or ¹²³I. Due to their small size, organic PET or SPECT labels are ideally suited for monitoring intracellular events, as they do not greatly affect the properties of the targeting device in general and its membrane transport in particular. Likewise, the azide moiety is small and can be used as an activator for intracellular imaging or therapy. Moreover, both components of the Staudinger reaction do not preclude crossing of the blood brain barrier and thus allow imaging or therapy of regions in the brain.

According to another embodiment the compounds and methods of the invention are used for targeted therapy. This is achieved by making use of a prodrug which comprises an azide and/or phosphine moiety and one or more pharmaceutically active agents (e.g. a drug). Suitable drugs for use in the context of targeted drug delivery are known in the art.

In yet another embodiment of the present invention, the use of a targeting moiety is replaced by selectively incorporating the azide or phosphine group that is a partner in the Staudinger reaction, into a target cell or tissue. This is achieved by using building block molecules such as metabolic precursor molecules, comprising, e.g. an azide reaction partner, that can be trapped or incorporated into biomolecules by the metabolism of the cell.

The pathways targeted in this way can be pathways that are common to all cells, such as DNA-, protein- and membrane synthesis. Optionally, these are metabolic pathways which are up regulated in disease conditions such as cancer or inflammation/infection. Alternatively, the targeted metabolic pathways are specific for a particular type of cell or tissue. The building blocks which can be used in the context of the present invention, include metabolic precursor molecules such as, but not limited to amino acids and nucleic acids, sugars, amino sugars, lipids, fatty acids and choline. Imaging of these compounds, such as amino acids, can reflect differences in amino acid uptake and/or in protein synthesis. A variety of sugars can be used for the labeling of carbohydrate structure. Fatty acids can be used for the labeling of lipids in e.g. cellular membranes.

Moreover, a number of analogs of metabolic precursors are known in the art, which can provide particular advantages for use in the context of the present invention. A non-limiting list of examples of metabolic pathways and corresponding metabolic precursors which can be labeled with azide or phosphine are provided below. Some of these become temporarily accumulated into the cell, while others are incorporated into biological macromolecules.

In a particular embodiment of this aspect of the invention a metabolic pathway, which is up regulated during a disease, like infection/inflammation or cancer, is targeted. Components which can be up regulated in disease conditions include for example DNA, protein, membrane synthesis and saccharide uptake. Suitable building blocks to label these elements include azide-labeled amino acids, sugars, nucleobases and choline and acetate. Cells with a high metabolism or proliferation have a higher uptake of these building blocks. Azide-derivatives can enter these pathways and accumulate in and/or on cells.

Accordingly the invention further relates to a kit for targeted medical imaging and/or therapeutics using drugs or imaging probes, comprising:

at least one building block comprising a Staudinger reaction partner; and at least one further probe selected from either;

an imaging probe comprising a Staudinger reaction partner and a label; or

a therapeutic probe comprising a Staudinger reaction partner and a pharmaceutically active compound.

wherein one of the building block or the imaging or therapeutic probe comprises, as Staudinger reaction partner, either at least one azide group and in that the other of the building block, imaging or therapeutic probe comprises at least one phosphine group, said phosphine and said azide groups being reaction partners for the Staudinger reaction.

A particular embodiment of the invention relates to the use of reporter probes, i.e. molecules which by their involvement in a cellular process, allow the visualization of a process or cell-type. Such a probe can make use of an endogenous mechanism of the cell, e.g. an endogenous enzyme for which a substrate is provided. Alternatively, such a probe functions by virtue of a foreign gene, referred to as a reporter gene. The reporter gene product can be an enzyme that converts a reporter probe to a metabolite that is selectively trapped within the cell. Alternatively, the reporter gene can encode a receptor or transporter or pump, which results in accumulation of the probe into the cells.

Fluorothymidine is a thymidine analog that is phosphorylated by thymidine kinase-1 (TK1), which can be used as a reporter gene, which results in cellular trapping. In cell culture, uptake correlates with TK1 activity and cellular proliferation.

According to a further embodiment of the invention, the reporter probe is a molecule which responds to a particular environment in a cell or tissue. Tissue hypoxia is central to the pathogenesis of cerebrovascular disease, ischemic heart disease, peripheral vascular disease, and inflammatory arthritis. It is also an ubiquitous feature of the growth of malignant solid tumors, where it bears a positive relationship to the aggressiveness of a tumor, and correlates negatively with the likelihood of response to chemotherapy or radiation therapy. Recent work has suggested that there is a common pathway of response to hypoxia in each of these settings. 2-Nitroimidazole compounds are reduced and trapped in hypoxic cells and can be used as sensors of oxygen tension in ischemic myocardium and tumors. Examples include fluoromisonidazole, fluoroerythronitroimidazole, azomycin-arabino side, vinylmisonidazole RP-170 (1-[2-hydroxy-1-(hydroxymethyl)-ethoxy]methyl-2-nitroimidazole) and SR 4554 (N-(2-hydroxy-3,3,3-trifluoropropyl)-2-(2-nitro-1-imidazolyl)acetamide). HL91 is a non-nitroimidazole compound that has a tumoral uptake Another suitable compound is diacetyl-bis(N-4-methylthiosemicarbazone)-copper(II) (ATSM).

Accordingly in a further aspect the invention relates to a kit for targeted medical imaging and/or therapeutics comprising:

at least one reporter probe comprising a Staudinger reaction partner; and at least one further probe selected from either;

an imaging probe comprising a Staudinger reaction partner and a label; or

a therapeutic probe comprising a Staudinger reaction partner and a pharmaceutically active compound,

wherein one of the reporter or the imaging or therapeutic probe comprises, as Staudinger reaction partner, either at least one azide group and in that the other probe comprises at least one phosphine group, said phosphine and said azide groups being reaction partners for the Staudinger reaction.

Optionally the primary targeting moiety or building block already comprises a detectable label. Preferably this label is different from the label that is introduced in a next step in the Staudinger reaction. Administration of the building block or primary targeting moiety with label such as FDG functionalised with azide, gives rise to an FDG like image, which may in a second step be overlayed with the image that is obtained from the activation step with a labeled phosphine. This combination of two imaging labels, one being present in the building block, reporter probe or primary targeting moiety and the other in the phosphine that is administered thereafter, has as potential advantages better target localization, artifact elimination, delineation of non relevant clearance and other pharmacokinetic pathways.

According to a particular embodiment of the invention, the compounds and methods described herein are used in vivo for the imaging or detection of tissues or cell types in the animal or human body. Alternatively, they can be equally used in vitro for the examination of biopsies or other body samples or for the examination of tissues which have been removed after surgery.

As described herein, according to a particular embodiment of the present invention, the composition (z) and the pro-drug or pro-imaging probe are provided sequentially, allowing the localization, optionally via binding of a target moiety of the composition (z) and optionally removal of the excess composition (z), before providing the label/imaging or prodrug compound. This ensures a higher signal to noise ratio in the image and/or a higher efficiency of the therapeutic and is generally referred to as ‘pre-targeting’ or ‘two-step’ targeting. According to a further embodiment the methods and compounds of the present invention are used for targeted signal amplification and/or polyvalency installation. Herein the composition (z) is preferably conjugated to a dendrimer, polymer or liposome containing multiple triphenylphosphine moieties. After receptor binding of the composition (z), preferably through a primary targeting moiety, a pro-imaging probe or prodrug comprising an azide conjugated to one or more MRI contrast agents, e.g. Gd chelates, is injected. The subsequent Staudinger reaction results in a high concentration of activated MRI contrast agent at the target tissue. Furthermore, the polyvalency at the target site will increase the reaction kinetics with the azide reporter conjugate (imaging probe), affording an efficient target activation of MRI contrast agent. Alternatively, the azide can also be comprised in the composition (z) as mentioned above and the triphenylphosphine conjugated to the pro-imaging probe or prodrug.

The probes and kits of the present invention are of use in medical imaging and therapy, more particularly ‘targeted’ imaging and therapy. The term ‘targeted’ relates to the fact that the pro-imaging probe or pharmaceutically active compound (prodrug) upon administration to the patient specifically interacts with or is introduced into a target molecule. This can be achieved according to the present invention by use of a targeting moiety or by use of a target metabolic substrate. Alternatively this can be obtained by providing a combined targeting and imaging or therapeutic probe (i.e. administration of these two components as a combined probe). This target molecule can be specific for a particular type of cell or tissue or can be common to all cells or tissues in the body.

The compositions of the invention can be administered via different routes including intravenous injection, oral administration, rectal administration and inhalation. Formulations suitable for these different types of administrations are known to the skilled person.

Prodrugs or pro-imaging probes according to the invention can be administered together with a pharmaceutically acceptable carrier. A suitable pharmaceutical carrier as used herein relates to a carrier suitable for medical or veterinary purposes, not being toxic or otherwise unacceptable. Such carriers are well known in the art and include saline, buffered saline, dextrose, water, glycerol, ethanol, and combinations thereof. The formulation should suit the mode of administration.

Hence in a further aspect the invention relates to a prodrug or pro-imaging probe comprising at least an azide and/or a phosphine group for use in the preparation of a medicament.

The invention is illustrated by the following non limiting examples.

### EXAMPLES

1) Triphenylphoshine conjugates are targeted to a disease site. After target binding and clearance from non-target tissue, an azido trigger-functionalized cascade-release dendrimer containing multiple FRET dyes is administered. Upon local activation in the Staudinger reaction with the targeted phoshine molecules, the dendrimer decomposes and releases multiple activated FRET dye molecules. Reference is made to FIG. 2 illustrating this example.
2) Azido trigger-functionalized cascade-release dendrimers containing multiple FRET dyes are targeted to a disease site. After target binding and clearance from non-target tissue, triphenylphosphine is administered systemically. Upon local activation by the phoshine molecules, the dendrimer decomposes and releases multiple activated FRET dye molecules. Reference is made to FIG. 3 illustrating this example.
3) As an alternative to example 2, the targeting moiety can be conjugated to the dendrimer via one of its tail ends instead of one FRET dye. Reference is made to FIG. 4 illustrating this example.
4) Activateable MRI contrast agents. The two highlighted carboxylic acids block two water coordination places in the inner sphere of the Gd chelate complex, which leads to a low relaxivity (thus, low signal intensity in MRI) of the construct. When the azide group reacts with a targeted triphenylphosphine moiety (A), the eliminating cascade initiated by the resulting amine affords the elimination of the appended carboxylic acids. The Staudinger reaction thus makes two coordination places available for water, which leads to a local signal increase. In B, the imaging compound is targeted instead of the activating compound. A targeted liposome with appended inactive Gd chelates is activated after reaction with systemic triphenylphosphine via the mechanism described for A. This leads to release of activated complexes. However, it is advantageous to keep the Gd complexes immobilized on the target of interest after activation. In C this is accomplished by attaching the lipid tail on a position that is not released by the azido trigger. The concept of the activateable probe can easily be applied in combination with a (targeted) cascade dendrimer, simply by mounting the basic structure of A on the tail ends of the constructs in examples 2 and 3 instead of the FRET dye. Reference is made to FIG. 5 illustrating this example. Also a large number of these constructs can be appended to a polymer for EPR targeting of the pro-imaging probes. In this respect, also a large number of activating molecules(/staudinger reaction partners) can be appended to such as polymers for EPR targeting, and subsequently reacted with systemic administered activateable MRI contrast agents.
5) In examples 1-3 the released substance is an activated dye. However, embodiments are possible where the released components comprise a mix between an activateable imaging probe (like a FRET dye) and one or more types of drugs, allowing imaging of drug activation. Furthermore, the targeting moiety itself and/or the imaging/therapy probe and/or composition (z) can also be labeled with an imaging agent (e.g. a dye of different wave length, or a radionuclide), allowing simultaneous imaging of drug delivery and activation.
6) The applications described in example (5) can also be performed using the activateable MRI contrast agent described in example (4) instead of other activateable imaging probes (like FRET) or in combination with other imaging probes.
7) Another embodiment concerning imaging of drug activation uses a profluorescent triphenylphosphine dye that is activated by the Staudinger reaction. After targeting of this dye to a disease site, an azide prodrug is administered (example A). This prodrug is then selectively activated at the disease site leading to release of active drug molecules as well as activation of the localized fluorescent probe.

In example B, the azide prodrug is targeted to the disease site. Subsequent administration of the profluorescent dye leads to local release of active drug molecules and activation of the dye.

Reference is made to FIG. 6 illustrating this example.

8) The synthesis of the model azido-prodrug 4-azidobenzyl N-benzyl carbamate (4) is described.
4-azidobenzyl alcohol (2):

A stirred solution of 4-aminobenzyl alcohol 1 (4.0 g, 32.5 mmol) in 60 ml 5M hydrochloric acid, was cooled to 4° C. and a solution of sodium nitrite (2.48 g, 35.9 mmol) in 20 ml water was added dropwise in 30 min. Sodium azide (8.50 g, 130.7 mmol) was added in small portions over 30 min. with vigorous stirring. The reaction temperature was kept under 5° C. After stirring for 1.5 hour at 4° C. the reaction mixture was poured into ice water and basified (NaHCO₃) to pH 8 (be aware of acid-base reaction). The water layer was extracted with EtOAc and washed with water (2×). The EtOAc layer was dried (MgSO₄) and evaporated. Light petroleum benzine was poured on the residue, stirred for 30 min. and kept at 0° C. overnight affording a cream-colored suspension. After filtration the residue was washed with 5 ml light petroleum benzine yielding 2 (3.93 g, 26.3 mmol, 81%) as a cream-colored powder (photosensitive). ¹H NMR, δ_{H} (CDCl₃) 1.72 (1H, s, OH), 4.67 (2H, s, CH₂), 7.02 (2H, d, J=8.48, aromatic), 7.35 (2H, d, J=8.67, aromatic).

4-azidobenzyl 4-nitrophenyl carbonate (3):

A solution of 4-nitrophenyl chloroformate (5.32 g, 26.4 mmol) and pyridine (4.38 g, 55.4 mmol) in 150 ml THF was stirred at 0-4° C., and a solution of 4-azidobenzyl alcohol 2 (3.93 g, 26.4 mmol) in 50 ml THF was added dropwise in 30 min. The reaction mixture was stirred in the dark at 25° C. for 70 hours. The THF was evaporated and 30 ml of EtOAc was added. The EtOAc layer was washed with water (2×), dried (MgSO₄) and evaporated. The residue was recrystallised from light petroleum benzine/EtOAc yielding 3 (6.26 g, 19.9 mmol, 75%) as a yellow powder. ¹H NMR, δ_{H} (CDCl₃) 5.19 (2H, s, CH₂), 7.00 (2H, d, J=8.48, azidobenzyl arom), 7.31 (2H, d, J=9.23, nitrophenyl arom), 7.37 (2H, d, J=8.67, azidobenzyl arom), 8.21 (2H, d, J=9.23, nitrophenyl arom).

4-azidobenzyl N-benzyl carbamate (4):

A solution of 4-azidobenzyl 4-nitrophenyl carbonate 3 (465 mg, 1.48 mmol) in 5.5 ml THF was cooled to 0° C. and after adding benzyl amine (267 mg, 2.49 mmol) and pyridine (50 mg, 0.63 mmol) to the stirred solution, the solution was allowed to warm up to rt and stirred for 18 hours. The THF was evaporated and 30 ml DCM was added. The organic layer was washed with water (1×), 0.1 M HCl (1×), water (1×), 0.1 M NaOH (1×) and water (1×). The organic layer was dried (MgSO₄), filtrated and the solvent was evaporated. Purification was performed by chromatography on silica gel, with DCM as eluent yielding 4-azidobenzyl N-benzyl carbamate 4 (347 mg, 0.123 mmol, 83%) as a slightly yellow solid. ¹H NMR, δ_{H} (CDCl₃) 4.36 (2H, d, J=6.03, CH₂ benzyl), 5.08 (2H, s, CH₂ azidobenzyl), 6.99 (2H, d, J=8.28, azidobenzyl arom), 7.25-7.35 (7H, m, aromatic). ¹³C NMR, δ_{H} (CDCl₃), δ5.59, 66.64, 119.53, 127.98, 129.12, 130.26.

Reference is made to FIG. 7 illustrating this example.

9) Activation of model azido-prodrug 4-azidobenzyl N-benzyl carbamate (4) by 3-(diphenylphosphino)benzenesulfonate (5) in two different media.

In DMF/H₂O (3.5/1): Prodrug 4 (5.3 mg, 18.8 μmol) was dissolved in 952 μl DMF-_{d7} and 275 μl D₂O. Phosphine 5 (12.1 mg, 33.2 μmol) was added and the reaction was followed with ¹H NMR. The reaction had run to completion within 14 hours as judged from the disappearance of the CH₂ azidobenzyl signal at 4.83 ppm and the appearance of the CH₂ signal of the corresponding aminobenzylalcohol (7) at 4.64 ppm.

In THF/H₂O (1/1): Prodrug 4 (4.8 mg, 17.0 μmol) was dissolved in 275 μl THF-_{d8} and 275 μl D₂O. Phosphine 5 (11.3 mg, 31.0 μmol) was added to the slightly yellow solution after which gas development was observed. The reaction was followed with ¹H NMR and had to run to 50% after 7 hours as judged from the disappearance of the CH₂ benzyl signal at 4.38 ppm and the appearance of the CH₂ signal of the corresponding free benzylamine (6) at 3.99 ppm.

Reference is made to FIG. 8 illustrating this example.

10) Synthesis of doxorubicin prodrug 9

Azidobenzyl carbamate Doxorubicin 9. A solution of 4-azidobenzyl 4-nitrophenyl carbonate 3 (20.6 mg, 65.6 μmol), Et₃N (9.6 μl, 69 μmol), doxorubicin (40 mg, 69 μmol) in DMF (4.8 ml) was stirred in the dark at RT under nitrogen gas for 20 h. After adding more Et₃N (9.6 μl, 69 μmol) the solution was stirred for another 24 h.

To the organic layer EtOAc/isopropanol 10% (10 ml) was added and the organic layer washed with water (1×). The red organic layer was dried (MgSO₄), filtrated and evaporated under reduced pressure (water bath was below the 30° C.). A red solid was collected (77 mg) and purified with preparative TLC (DCM/MeOH 9/1). The collected fraction at R_{f} 0.72 was extracted with EtOAc. The organic layer was evaporated under reduced pressure and ACN/water 1/1 was added for freeze-drying. After freeze-drying a red solid (31.8 mg, 67%) was collected. ¹H NMR δ_{H} (CDCl₃) 1.29 (3H, d, J=6.6, C—CH₃) 1.80-1.90 (2H, m, CH₂ carbohydrate), 2.17 (1H, dd, J_{A}=14.8, J_{B}=4.0, CH₂), 2.33 (1H, br dt, J_{A}=14.7, J_{B}=1.7, CH₂), 3.02 (1H, d, J=19, CH₂), 3.28 (1H, d, J=19, CH₂), 3.66 (1H, br s, CH carbohydrate), 3.86 (1H, m, CH carbohydrate), 4.09 (3H, s, O—CH₃), 4.14 (1H, q, J=6.4, CH—CH₃) 4.76 (2H, s, CH₂—OH), 4.99 (2H, s, CH₂ linker), 5.13 (1H, d, J=8.6, amide NH), 5.29 (1H, br m, CH), 5.50 (1H, d, J=3.5, CH carbohydrate), 6.97 (2H, d, J=8.3, aromatic azide), 7.29 (2H, d, J=8.3, aromatic azide), 7.40 (1H, dd, J_{A}=8.5, J_{B}=0.75, aromatic dox), 7.80 (1H, dd, J_{A}=8.5, J_{C}=7.7, aromatic dox), 8.05 (1H, dd, J_{B}=0.75, J_{C}=7.7, aromatic dox). MALDI TOF MS: m/z: 742 [M+Na]⁺.

Reference is made to FIG. 9 illustrating this example.

11) Activation of the doxorubicin prodrug (9) followed with HPLC and LC-MS

The activation was carried out in the following way: a solution of prodrug in water or growth medium was mixed with a solution of triphenylphosphine-3,3′,3″-trisulfonic acid trisodium salt in water or growth medium. Samples were taken from the reaction mixture (at 37 C) every 2 hours and measured in the HPLC or LCMS. Several different concentrations and ratios prodrug/triphenylphosphine were used. The first activation experiment was done in water with a concentration of 100 μM for the prodrug and 200 μM for the phosphine, and monitored with HPLC. After 8 hours 15% doxorubicin had formed and the reaction was complete after 20 hours (FIG. 10).

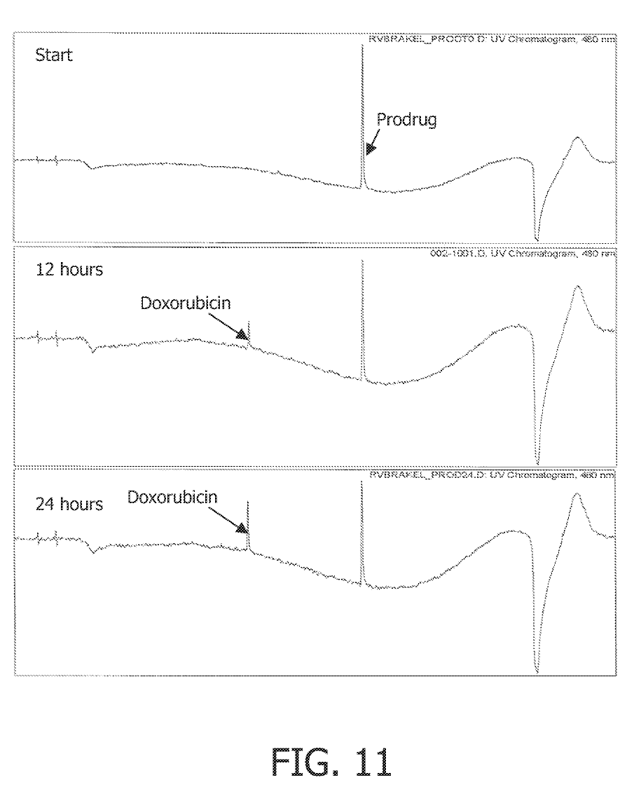

The second activation was done at a 10-fold lower concentration, 10 μM for the prodrug and 20 μM for the phosphine, and monitored with LCMS. After 24 hours the reaction had run to approximately 30% (FIG. 11).

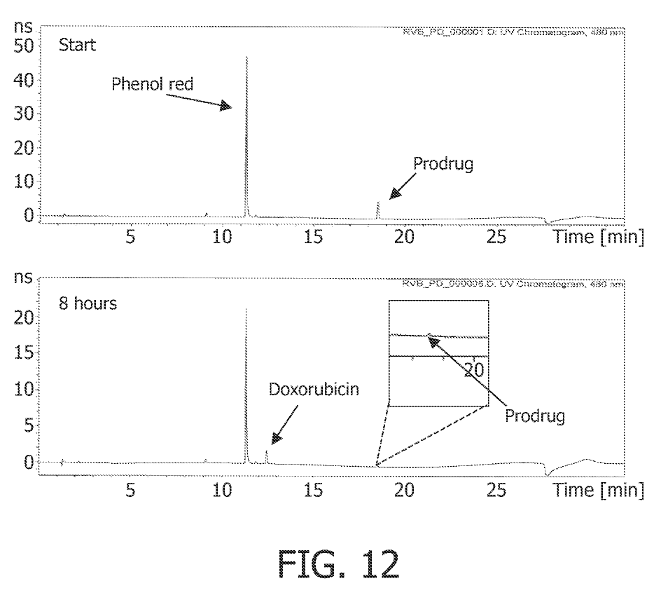

The third and last activation was done in cell growth medium at 10 μM prodrug. Since the triphenylphosphine is slowly oxidized in cell growth medium, the protocol was adjusted. During this experiment a fresh portion (60 μM) triphenylphosphine was added to the mixture twice a day. After 8 hours the reaction had run to 85% (FIG. 12).

Reference is made to FIGS. 10-12 illustrating this example.

12) Cell proliferation assay using A431 cells with prodrug 9 (pro-dox) in situ activated by triphenylphosphine.

The in vitro anti-cancer activity of prodrug 9 (pro-dox) was assessed by means of cytotoxicity assays. For these cellular experiments the A431 cell line was used. This cell line is derived from a human vulvar skin squamous cell carcinoma (SCC). The addition of the prodrug in combination with phosphine should lead to a reduction in proliferation compared to the addition of pro-dox without phosphine, due to the release of doxorubicin by means of the staudinger reaction.

### Cells and Reagents

Cells were maintained at 37° C. in DMEM (InVitrogen), supplemented with 10% heat-inactivated fetal bovine serum and 0.05% glutamax (InVitrogen) in the presence of penicillin and streptomycin.

Doxorubicin (Toronto Research Chemicals) and pro-dox (9) were, prior to each experiment, freshly dissolved in DMF at 10 mM and subsequently serial diluted in pre-warmed culture medium. Triphenylphosphine-3,3′,3″-trisulfonic acid trisodium salt (Sigma) was dissolved in PBS at 10 mM and serial diluted in pre-warmed medium. Methylthiazolyldiphenyl-tetrazolium bromide, MTT (Sigma) was used as described by the manufacturer.

### Proliferation Assay

Cells were plated out in a 96 well plate (Nunc) at a density of 7500 cells/well, followed by addition of reagents the next day. After 72 hours incubation cell proliferation was assessed by means of a MTT assay. Briefly, MTT was dissolved in pre-warmed medium at 5 mg/ml, passed through a 0.22 μm filter and 50 μl was added to each well. Following an incubation period of 120 minutes the medium was gently aspirated. The formed formazan crystals were dissolved in 100 μl DMSO and the absorbance, or optical density (O.D.), was measured with a plate reader (BMG) at 560 nm. The inhibition of proliferation was determined by reporting the O.D. of the treated group (T) with the O.D. of the appropriate control group (C), and expressed as [(1-T/C)×100%].

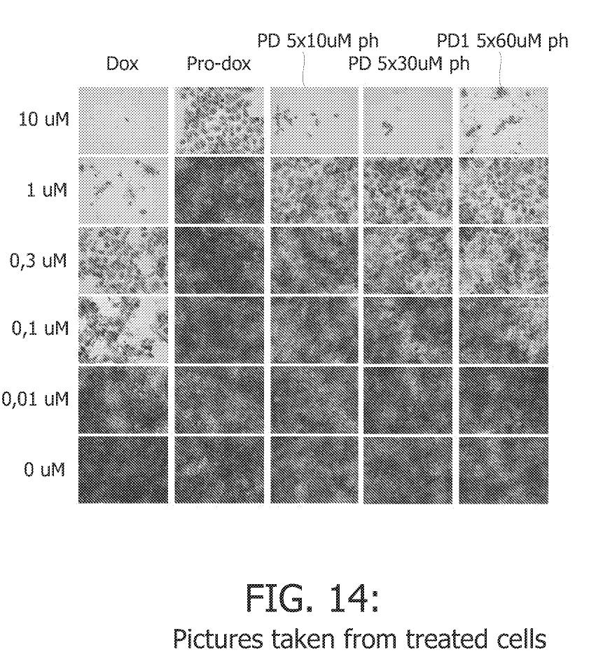

As mentioned in Example 11, the phosphine is slowly oxidized in cell growth medium. Therefore, instead of adding the prodrug to the cell culture together with a single batch triphenylphosphine on day 2, the phosphine was added at fixed intervals from day 2 to day 4. Three phosphine dosing schemes were investigated: 5×10 μM, 5×30 μM, and 5×60 μM. The range of the concentrations used for the doxorubicin drugs was 0-0.01-0.1-0.3-1.0-10 μM. The results in FIGS. 13 and 14 show the lowest anti-proliferative activity for pro-dox, followed in order of increasing activity by pro-dox activated by 10, 30, and 60 μM repetitive doses of phosphine. Doxorubicin itself was found to have the highest activity, 2 orders of magnitude higher than its masked inactive pro-dox analog. Although the pro-dox/phosphine combination does not equal the parent drug in activity, treating the cells with pro-dox with the 60 μM repetitive dose of phosphine still results in a 33-fold increase of activity over pro-dox alone.

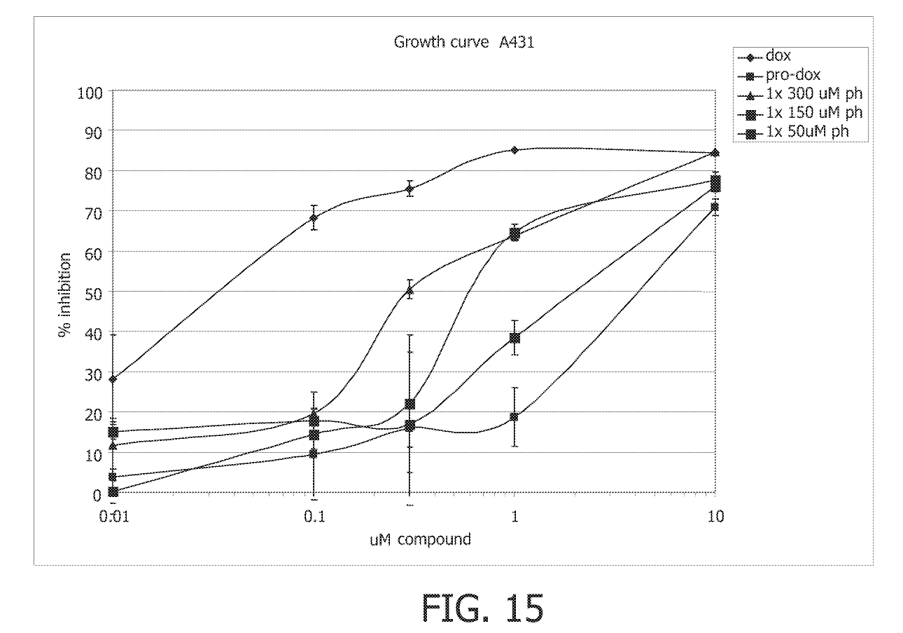

The above phosphine-dosing scheme was compared to a single batch-dosing scheme at the same total amount of phosphine. The results of this experiment are depicted in FIG. 15. From the figure is becomes clear that, at identical total phosphine amounts, a single dose of phosphine is not nearly as effective in activating the doxorubicin prodrug as the sequential additions of phosphine.

Reference is made to FIGS. 13-15 illustrating this example.

1. A method for preparing and activating a prodrug or a pro-imaging probe comprising the steps of:
a) functionalising a drug (x) or imaging probe (y) with at least one azide and/or phosphine group to create a prodrug or pro-imaging probe;
b) reacting the prodrug or pro-imaging probe by a Staudinger reaction with a composition (z) comprising at least one azide and/or phosphine group as a reaction partner in the Staudinger reaction,
thereby activating the drug or imaging probe.

2. A method for preparing and activating a prodrug or a pro-imaging probe comprising the steps of:
a) functionalising a drug (x) with at least one azide and/or phosphine group to create a prodrug;
b) functionalising an imaging probe (y) with at least one azide and/or phosphine group which is a partner in the Staudinger reaction for the azide and/or phosphine group of the prodrug of step (a) to create a pro-imaging probe;
c) reacting the prodrug and pro-imaging probe by a Staudinger reaction,
thereby activating the drug and/or imaging probe.

3. A kit for medical imaging and/or therapeutics, comprising:
at least one prodrug (x) and/or pro-imaging probe (y), comprising at least one azide and/or phosphine group;
a composition (z) comprising at least one azide and/or phosphine group capable of reacting with the prodrug or pro-imaging probe in a Staudinger reaction to form a drug or imaging probe.

4. The kit according to claim 3, which comprises at least one prodrug.

5. The kit according to claim 3 comprising a prodrug and a pro-imaging probe.

6. The kit according to claim 5 wherein the prodrug and the pro-imaging probe comprise a phosphine group.

7. The kit according to claim 3 wherein at least one of the prodrug (x), pro-imaging probe (y) or composition (z) comprises a primary targeting moiety.

8. The kit according to claim 7, wherein the targeting moiety binds to a receptor.

9. The kit according to claim 7, wherein the targeting moiety is an antibody.

10. The use of a prodrug or pro-imaging probe comprising an azide and/or a phosphine group, said phosphine and/or said azide groups being suitable reaction partners for the Staudinger reaction, as a tool in medical imaging.

11. The use of a pro-imaging probe comprising an azide and/or a phosphine group, said phosphine and/or said azide groups being suitable reaction partners for the Staudinger reaction, in the manufacture of a tool for medical imaging.

12. The use of a prodrug comprising an azide and/or phosphine group and a detectable label, said phosphine and/or azide groups being suitable reaction partners for the Staudinger reaction, in the manufacture of a tool for medical imaging.

13. A kit for targeted medical imaging and/or targeted therapeutics comprising:
at least one building block comprising a Staudinger reaction partner; and at least one further probe selected from either:
an imaging probe comprising a Staudinger reaction partner and a label; or
a therapeutic probe comprising a Staudinger reaction partner and a pharmaceutically active compound,
characterized in that one of the building block or the imaging or therapeutic probe comprises, as Staudinger reaction partner and activator respectively, either at least one azide group and in that the other of the building block, imaging or therapeutic probe comprises at least one phosphine group, said phosphine and said azide groups being reaction partners for the Staudinger reaction.

14. A kit for targeted medical imaging and/or targeted therapeutics comprising:
at least one reporter probe comprising a Staudinger reaction partner; and at least one further probe selected from either:
an imaging probe comprising a Staudinger reaction partner and a label; or
a therapeutic probe comprising a Staudinger reaction partner and a pharmaceutically active compound,
characterized in that one of the reporter or the imaging or therapeutic probe comprises, as Staudinger reaction partner, either at least one azide group and in that the other probe comprises at least one phosphine group, said phosphine and said azide groups being reaction partners for the Staudinger reaction.

15. A kit for targeted medical imaging and/or targeted therapeutics comprising:
at least one prodrug (x) comprising at least one azide and/or phosphine group;
at least one pro-imaging probe (y) comprising at least one azide and/or phosphine group capable of reacting with the prodrug in a Staudinger reaction to form a drug and an imaging probe.

16. Prodrug comprising at least a phosphine group for use in the preparation of a medicament.

17. Pro-imaging probe comprising at least an azide and/or phosphine group for use in the preparation of a medicament.
